(19) United States
(12) Patent Application Publication
(10) Pub. No.: US 2009/0155339 A1
(43) Pub. Date: Jun. 18, 2009

(54) Biocompatible Antimicrobial Filament Material

(75) Inventors: Sven Eggerstedt, Hamburg (DE); Erich K. Odermatt, Schaffhausen (CH); Rainer Bargon, Mengen (DE)

Correspondence Address: THE NATH LAW GROUP, 112 South West Street, Alexandria, VA, 22314, US

(73) Assignee: AESCULAP AG & CO. KG, Tuttlingen (DE)

(21) Appl. No.: 11/992,012

(22) PCT Filed: Sep. 15, 2006
(86) PCT No.: PCT/EP2006/009003

§ 371 Date: Oct. 1, 2008

(30) Foreign Application Priority Data
Sep. 15, 2005 (EP) PCT/EP2005/009917

(51) Int. Cl. A61K 9/70; A61P 31/00

(52) U.S. Cl. 424/443

(57) ABSTRACT

The invention provides a biocompatible filament material having an antimicrobial finish, in particular in the form of a surficial layer, the finish comprising polyhexamethylenebiguanide (PHMB) as a nonspecifically antimicrobially active component, and also a process for producing the filament material, comprising the steps of:
producing an active solution from the nonspecifically antimicrobially active component PHMB and a solvent,
transferring PHMB from the active solution onto and/or into the filament material, and
drying the filament material comprising the transferred PHMB.

The present invention relates to a biocompatible filament material having an antimicrobial finish and also to a process for producing the filament material and to providing the filament material for use in the human or animal organism.

At present, countless interventions for diagnostic or therapeutic purposes are performed every day on the human or animal body. In these interventions, various implants, especially filament materials, are temporarily or permanently introduced into the body, examinations are performed using probes remaining in the body for a certain length, catheters are used to supply or remove substances, and various wounds are sealed following medical interventions or injuries. With each of these interventions there is an appreciable risk of wound infection due to pathogenic microbes being imported into the body. Especially in the case of absorbable filament materials, for example for wound closure, where no further intervention for removing the absorbed foreign body is intended, it is extremely important that the wound, the material and also the environment of the material remain free of infections until the material is fully absorbed and the wound is healed. Especially within the first two weeks following an intervention there is a heightened risk of infection due to unwanted imported pathogens and multiplication of pathogenic microbes due to the weakened immune defense and the wound-healing process.

Many attempts have therefore already been made to preempt complications due to pathogenic microbes by endowing implants with active compounds which augment the natural immune defense and kill the imported pathogenic microbes or at least hinder their growth. For instance, U.S. Pat. No. 4,024,872 (U.S. Pat. No. 3,991,766, U.S. Pat. No. 6,528,107) and also WO 03/009879 disclose respectively the impregnation and the disposition of a reservoir with a broad-spectrum antibiotic. EP 350147, U.S. Pat. No. 5,091,442, WO 03/009879, US 2002/0193879 and also WO 96/22114 are just some documents which describe the use of triclosan in the form of coatings or adjuncts for the manufacture of polymeric medical device materials. The medical devices described therein all utilize either specific antibiotics or broad-spectrum antibiotics or the widely used antimicrobial triclosan for controlling pathogenic microbes. The disadvantage of these actives consists in the generally known development of resistances of the pathogenic microorganisms to the antibiotics used that has come to be greatly feared in hospitals.

Triclosan, previously considered to have a nonspecific mechanism of action, was shown at the end of the 1990s to have a specific mechanism of action whereby it intervenes in the fatty acid synthesis of bacteria. This specificity of action on the part of the active compound triclosan creates the risk that bacterial strains exposed to non-bactericidally active concentrations of triclosan, as used in particular to coat temporarily used products such as catheters, gloves or the like, will develop resistance. There is also the risk of cross-resistances developing whereby the bacteria can as a result also become resistant to certain antibiotics.

It is an object of the present invention to provide a biocompatible filament material having a nonspecific but yet highly active antimicrobial finish which effectively prevents colonization by pathogenic microbes and also prevents the development of resistance to the antimicrobial finish.

We have found that this object is achieved by a biocompatible filament material having an antimicrobial finish, in particular in the form of a surficial layer, the finish comprising polyhexamethylenebiguanide (PHMB) as a nonspecifically antimicrobially active component. The filament material including the antimicrobial finish is preferably present in dry form.

The advantage of the filament material provided with PHMB is the nonspecific mechanism of action of PHMB with regard to pathogenic microbes which forecloses the risk of resistance developing to the active component PHMB. PHMB is highly compatible and is used in swimming pool water treatment without undesirable side effects on the human organism being known. The present invention's filament material with an antimicrobially preferably surficial layer comprises polyhexamethylenebiguanide (PHMB) as nonspecifically antimicrobially active component.

In one embodiment of the present invention, the antimicrobial finish as well as PHMB comprises further specific or nonspecific antimicrobial components. Preferably, the filament material of the present invention comprises exclusively polyhexamethylene-biguanide as nonspecifically antimicrobially active component.

The filament material is preferably present in not more than two-dimensional form. Its configuration is preferably one-dimensional or two-dimensional.

The filament material may comprise in particular a mono- or multifil material, in which case a multifil filament material is preferred. In accordance with the present invention, the filament material may comprise a braid, a formed-loop knit or a drawn-loop knit, in which case a formed-loop knit is preferred.

In a further embodiment, the filament material is in the form of a textile sheetlike construction, in particular as a tape or mesh, for example as a hernial mesh, the form of a tape being particularly preferred. It is particularly preferred for the filament material to comprise suture.

In a further embodiment of the present invention, the nonspecifically antimicrobially active component PHMB is present on the surface of the filament material. Advantageously, the filament material has an antimicrobial impregnation.

In a further preferred embodiment, the nonspecifically antimicrobially active component is present in the filament material and preferably in the outer layer of the surface. The nonspecifically antimicrobially active component PHMB is advantageously incorporable by means of solvents in layers underneath the surface of preferably swellable filament materials.

It is advantageous for the PHMB to be free of covalent bonds to the filament material. In one particular embodiment, PHMB can form ionic bonds to anionic, i.e., negatively charged, materials.

In a further execution of the invention, the PHMB is freely available and is in particular capable of diffusion into the surrounding body tissue. Depending on the strength of the bond between the PHMB and the filament material, the PHMB can be releasable from the filament material by washing out.

Preferably, the filament material is antimicrobially active for at least 7 days, preferably for 10 to 14 days.

It may further be desired according to the present invention for the filament material to comprise a bioabsorbable material. This is achievable in particular when the filament material comprises in vivo hydrolyzable polymers. Advantageously, the in vivo hydrolyzable polymers comprise polymers based on lactide, glycolide, trimethylene carbonate (TMC) and/or dioxanone, a filament material composed of lactide and/or glycolide polymer, preferably composed of glycolide polymer, being particularly preferred. In accordance with the present invention it can thus be provided that the active compound polyhexamethylene-biguanide PHMB is freely available, and diffusible into tissue, following complete absorption or after absorption of the surficial layers of the filament material. In accordance with this particular embodiment, PHMB is in its radius of action not restricted to the surface of the filament material of the present invention, but is also antimicrobially active in the immediate environment of the filament material.

In another embodiment, the filament material comprises a nonabsorbable material, in particular a polymer, preferably polyethylene and/or polypropylene. It is also possible for the filament material to comprise partly absorbable material.

Advantageously, the PHMB content on the surface of the finished filament material is between 0.1 and 100 μg/cm². In the case of PHMB present in the filament material, the PHMB content in the antimicrobially finished filament material, in particular in a polymeric filament material, is advantageously 2-3000 ppm.

In a further execution, the filament material has a surface coating other than the antimicrobial finish. The surficial coating can be absorbable or nonabsorbable, in which case an absorbable coating is preferred. Advantageously, the absorbable coating comprises a bioabsorbable polymer, in particular an in vivo hydrolyzable polymer. For further details reference is made to the above description.

Advantageously, a coating for enhancing the lubricity of the filament material, in particular suture, can be provided to facilitate handling. It is possible for a nonabsorbable filament material to have a coating of absorbable material. The absorbable coating material can be used to control the delivery and diffusion of the nonspecifically antimicrobially active component PHMB from the surface of the filament material into the surrounding tissue since the PHMB only becomes active after absorption of the coating material.

In another embodiment, the coating material comprises a nonabsorbable material. The coating of nonabsorbable material is advantageously provided over the entire area of the coating with through openings for retarded and controlled delivery of the nonspecifically antimicrobially active component PHMB into the tissue in order to ensure contact between the nonspecifically antimicrobially active component on or in the filament material and the surrounding tissue.

Advantageously, the fraction of coating material in terms of total material (filament material including coating material) is in the range from 0.1% to 5% by weight, which corresponds to the range from 1000 ppm to 50 000 ppm.

The coating material preferably comprises at least one material selected from the group consisting of polyethylene, polyester, silicone and polyurethane. The coating material consists more preferably of absorbable materials comprising lactide, glycolide, trimethylene carbonate (TMC), ε-caprolactone (ECL) or dioxanone, which may each be present as monomer or as polymer.

In another development of the present invention, the filament material is free of envelopments and the nonspecifically antimicrobially active component PHMB is directly present on the product surface.

Preferably, about 60% of the PHMB is directly releasable after use of the filament material, whereas about 40% of the PHMB is still bound in the material even after 14 days and so the filament material continues to have an antimicrobial activity directly at its surface.

In a preferred embodiment of the present invention, the PHMB is at least partly present in a form which is sparingly soluble in water or in an aqueous physiological medium. Using sparingly soluble PHMB makes it possible to prolong the release time of PHMB from the filament material or its surface, so that the antimicrobial effect continues to last for a period of more than 14 days. At the same time, a retarded delivery of PHMB can be used to keep its cytotoxic effect to a minimum. Combinations of various forms of PHMB are particularly beneficial. It is known that PHMB can have different molecular weights. It is also known that PHMB can be present in the form of derivatives which are readily soluble, less soluble or sparingly soluble in water. Mixtures of at least two PHMB forms are particularly beneficial. The antiseptic effect and its duration can thereby be adjusted to specific values. Preference is given to a combination of at least one PHMB variant in a sparingly soluble form and at least one PHMB variant in a relatively soluble form. For instance, the readily soluble form can have diffused into the filament material or into the individual fibres, whereas the sparingly soluble form has come to be deposited on the surface. By choosing suitable solvents, in particular organic solvents, it is possible to produce suitable measurements or solvent mixtures which are in the desired concentration range in particular. Suitable sparingly soluble forms of PHMB are fatty acid salts having an even-numbered unbranched fatty acid radical of 8 to 20 carbon atoms. The solubility of sparingly soluble PHMB compounds is preferably less than 1 g/l of water, preferably less than 0.1 g/l of water.

When the filament material is composed of synthetic organic polymers, which is preferred, then it is advantageous to choose the particular solvent such that the filament material is not dissolved. However, swelling can be advantageous in many cases.

The present invention further provides a process for producing a filament material in accordance with the present invention, comprising the steps of:
producing an active solution from the non-specifically antimicrobially active component PHMB and a solvent,
transferring PHMB from the active solution onto and/or into the filament material, and
drying the filament material comprising the transferred PHMB.

The solvent may comprise in particular at least one organic solvent, for example an alcohol, a ketone or an aromatic solvent. Particular preference is given to the solvents isopropanol, ethyl acetate, toluene and/or xylene, of which ethyl acetate is particularly preferred. In a further embodiment of the process according to the present invention, the solvent comprises water. It is similarly possible to use mixtures of various solvents, especially of the solvents just recited, with each other or with water.

The concentration of PHMB in the active solution depends on the purpose of the nonspecifically antimicrobially active component. In the case of a bactericidal effect, a higher concentration is preferred than in the case of a bacteriostatic effect on the part of the PHMB. Preferably, the concentration of PHMB in the active solution is in the range from 0.1% to 20% by weight and especially in the range from 0.3% to 8% by weight.

In a particularly preferred embodiment, the transferring of the nonspecifically antimicrobially active component PHMB is effected by dipping the filament material into the active solution. Alternatively, it can be advantageous to effect the transferring by spraying the active solution onto the filament material.

The process of the present invention preferably comprises a fully automated process, in particular a fully automated coating process. In particular a filament material present as suture can be transported from an unwinding package preferably through a dipping eyelet in a dip bath between two squeeze-off rolls whose squeeze pressure can be varied. The suture can subsequently be led over a plurality of mobile metal rollers and be dried in particular by means of IR radiation and convection dryer. Before being wound up as a thread, the suture preferably passes through a traversing unit. The thread transport is advantageously engineered to a constant thread tension during the entire winding operation.

The application process for the active solution is dependent on the geometry and on the material's constitution, in particular the absorbency of the filament material. For geometrically bulky sterical shapes it will be found advantageous to spray the filament material repeatedly, if necessary, with the active solution from all spatial directions, whereas in the case of absorbent filament materials it is preferable to perform a dip process to drench the filament material with the active solution.

In a particular development of the process, a swellable filament material is immersed in the active solution and left in the solution until the filament material has swollen to the desired depth of penetration. The PHMB penetrates with the solvent into the interior of the filament material. Subsequently, the solvent is removed by drying preferably at room temperature or, if appropriate, at higher temperature and/or reduced pressure, and the PHMB remains in the formerly swollen layers of the filament material even after the solvent has been removed.

In a further embodiment of the process, the active solution may also comprise a suspension or emulsion of PHMB in a suitable suspension or emulsion medium with which the filament material of the present invention is provided by the process described above.

In a particular embodiment, the nonspecifically antimicrobially active component PHMB is dissolved together with the coating material of the present invention and subsequently applied to the filament material. This means that the antimicrobial finish is like the coating of the filament material possible in one operation and at the same time a delivery of the nonspecifically antimicrobially active component PHMB to the surrounding tissue is ensured immediately after deployment of the filament material. In the event that the coating is performed after the nonspecifically antimicrobially active component, it is particularly advantageous for the coating material to be absorbable, so that the antimicrobially active (PHMB) can be released after absorption of the coating.

Depending on the application process for the active solution, the concentration of coating material in the solution can vary. It is advantageous to choose a higher active concentration for a single application of the active solution than for a very long exposure of the filament material, in particular in the case of immersion into the active solution. Advantageously, the concentration of coating material in the active solution is in the range from 0.5% to 5% by weight and preferably in the range from 1% to 3.5% by weight.

To ensure good adhesion of the nonspecifically antimicrobially active component PHMB on the filament material or good penetration of the active solution into the filament material, the filament material can be surface treated before the antimicrobial solution is applied. Preferably, the filament material is subjected to a surface treatment, in particular a sputtering operation or plasma activation, before the antimicrobial finish, and a plasma treatment is particularly preferred. Plasma activation preferably utilizes the atmospheric pressure plasma liquid deposition (APPLD) process. The surface treatment improves the absorbency and/or adhesion, in particular of the nonspecifically antimicrobially active component PHMB, on the surface of the filament material.

The present invention further comprises provision of a filament material in accordance with the present invention for use in relation to the human or animal organism. The filament material can be used both internally and externally. Preference is given to provision for an internal use in the form of a suture, tape or mesh, in particular a hernial mesh, in the human organism. The filament material of the present invention is preferably used for wound closure and/or in the case of hernias.

### FIGURE DESCRIPTION

FIG. 1: Functional diagram of a thread-coating range:
From an unwinding package 1 the thread 2 travels through a dipping eyelet 3 into a dip bath 4 between two mobile squeeze-off rolls 5 whose squeeze pressure can be varied. The thread 2 is led over a plurality of mobile metal rollers 6 and dried by means of IR radiator 7 and convection dryer 8. The thread passes through a traversing unit 9 before arriving at the winding package 10.

FIG. 2: Scanning electron micrograph of a Safil® thread coated with PHMB-HCl

FIG. 3: Scanning electron micrograph of a Safil® thread coated with PHMB stearate

Further features of the invention will be apparent from the following description of preferred embodiments in the form of examples in connection with the subclaims. Individual features of the invention can be actualized alone or in combination with one another. The embodiments described merely serve to elucidate and to better understand the invention and are in no way to be understood as restricting.

### Material and Methods

Nitroprusside (disodium pentacyanonitrosyl(II) dihydrate), potassium hexacyanoferrate(II), sodium hydroxide, sulfuric acid (1 M), hydrogen peroxide (Perhydrol, 30%), stearic acid and iron(II) sulfate were obtained from Merck KGaA (Darmstadt), borate buffer (pH=11) from Riedel-de-Haen (Seelze) and polyhexamethylenebiguanide (short: PHMB) obtained from Arch Chemicals GmbH (Ratingen) as 20% solution and diluted down to 15%, 10%, 5% and 1% for the experiments. Safil beige USP 2/0 from B. Braun was used as suture.

The coatings were carried out using a fully automatic coating range (see FIG. 1).

The scanning electron micrographs were taken with a Zeiss (Oberkochen) scanning electron microscope of the type 435 VP.

Microbial analyses were carried out at medical device testing GmbH (Ochsenhausen) for PHMB-HCl coatings and at Medical Device Service Dr. Rossberger GmbH (Gilching) for the PHMB stearate coatings.

Cytotoxicity tests were carried out at Medical Device Service Dr. Rossberger GmbH (Gilching) for the PHMB-HCl coatings and at NAMSA (United States) for the PHMB stearate coatings.

Coating can be carried out by covering the multifilament, without penetration of the coating, by using an aqueous or organic suspension of a sparingly soluble PHMB salt, and/or by a penetration of the coating to the interior of the multifilament taking place, in which case the individual filaments are sheathed with the coating.

### EXAMPLE 1

### PHMB-HCl

### 1.1 Analysis of PHMB-HCl Solutions

The analysis of PHMB has already been described in the literature (H. Bratt, D. E. Hathway “Characterization of the Urinary Polymer-related Materialform Rats given Poly[biguanidine-1,5-diylhexamethylene hydrochloride]”, Makromol. Chem. 1976, 177, 2591-2606). The reagent solution was prepared as follows:

10 mL of nitroprusside solution (10%), 10 mL of potassium hexacyanoferrate(II) solution (10%) and 10 mL of aqueous sodium hydroxide solution (10%) and 30 mL of water were mixed and stored in the fridge overnight. The reagent solution cannot be stored for several days.

### 1.2 Analysis of PHMB Solutions

To measure the PHMB concentration in solution, 1 mL of reagent solution was admixed with 5 mL of sample. Instead of heating to 45-55° C., as described in the literature, the reaction was completable by 5 minutes of sonication in an untrasonic bath. The absorption was determined at 530 nm with a spectrophotometer.

### 1.3 Analysis of PHMB on Glycolic Acid Threads

To analyze the PHMB on the thread, it is first necessary to destroy the glycolic acid and any D&C violet No. 2 dye in the thread as interfering components. This was done as follows:

6 cm of thread were placed in a 25 mL graduated flask and admixed with 3 mL of sulfuric acid (1 M) and 3 mL of hydrogen peroxide (Perhydrol). The samples were sonicated in an untrasonic bath at 70° C. for 4 h and then left in the bath for some hours until the bath had reached ambient temperature. Aqueous sodium hydroxide solution is added for neutralization (pH=7) and, to destroy excess glycolic acid, 0.1 mL of an iron(II) sulfate solution (0.1 M) is added and the flask is ultrasonicated for a further 4 h. The solution is made up to 25 mL with borate buffer (pH=11). 1 mL of the above solution was then admixed with 0.2 mL of reagent and with 1 mL of borate buffer and measured.

1.4 Coating of Thread with PHMB-HCl Solution

A coating range (cf. also FIG. 1) was used to continuously coat about 100 m of a Safil® beige thread (polyglycolic acid) of USP 2/0 gauge. The coating used was a 20% aqueous PHMB solution. The squeeze pressure of the squeeze-off rolls was 100 N, the coating speed was 5 m/min and the drying temperature was 350° C.

Analytical determination from 5 measurements showed that 1786±143 mg/m² of PHMB-HCl were left on the thread.

Depending on the coating concentration, coating contents of 90-130 mg/m² were obtained on the threads.

### 1.5 Surface Examination

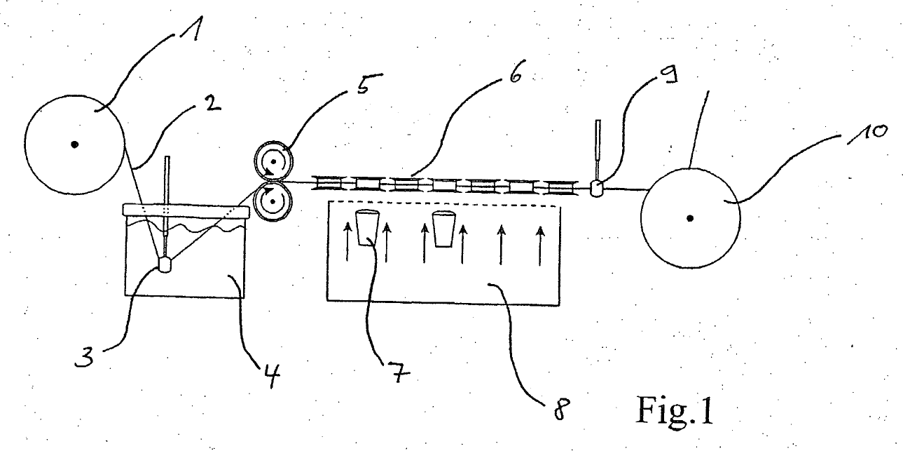

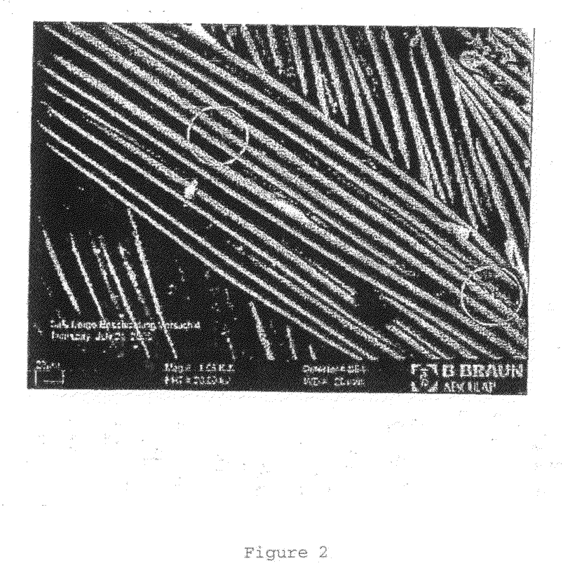

Electron micrographs were taken for surface examination. The micrographs were obtained with an acceleration voltage of 20 kV, a working distance of 25 mm and through detection of secondary electrons at 1060-fold magnification. The circles indicate coating material (cf. also FIG. 2).

The coating appears as a granular plaque (cf. also FIG. 2). These are distinctly visible in the interstices. Deposition is relatively uniform.

### 1.6 Antimicrobial Efficacy of PHMB-HCl

Antimicrobial efficacy was determined according to a method described in the European Pharmacopeia 2004. To this end, 1.00 gram of Safil of USP 2/0 thread gauge, which was coated with PHMB solution, was transferred into 5 ml of physiological saline solution. The solution was then inoculated with 10⁶ CFUs (colony-forming units) per g of suture material, suspended and after one hour, after 24 hours and after 72 hours of exposure time at room temperature the microbe numbers in the suspension were determined. To this end, 0.5 ml was removed per time, per microbe and per concentration and for each 100 μl and 10 μl were directly plated out on agar plate and the remainder was membrane filtered. Microbe number determination after incubation was done on agar plates at high microbe counts (>200 CFUs) and by the method of membrane filtration when microbe counts were expected to be low.

TABLE 1
Safil beige USP 2/0 coated with 15% PHMB-HCl solution

| Time | P. aeruginosa | MRSA | C. albicans | MRSE | E. coli |
|---|---|---|---|---|---|
| (days) | ATCC 9027 | ATCC 33592 | ATCC 10231 | CIP 105810 | ATCC 8739 |
| MC | 1.20 × 10⁶ | 1.60 × 10⁶ | 4.00 × 10⁵ | 2.60 × 10⁶ | 7.60 × 10⁶ |
| (inoculum) | | | | | |
| MC after | 2 | 9 | 2 | 15 | 17 |
| 1 h | | | | | |
| MC after | 0 | 0 | 0 | 6 | 0 |
| 24 h | | | | | |
| MC after | 0 | 0 | 0 | 0 | 0 |
| 72 h | | | | | |
| Log₁₀ | 6.1 | 6.2 | 5.6 | 6.4 | 6.9 |
| reduction | | | | | |

MC = microbe count

TABLE 2
Safil beige USP 2/0 coated with 10% PHMB-HCl solution

| Time | P. aeruginosa | MRSA | C. albicans | MRSE | E. coli |
|---|---|---|---|---|---|
| (days) | ATCC 9027 | ATCC 33592 | ATCC 10231 | CIP 105810 | ATCC 8739 |
| MC | 1.20 × 10⁶ | 1.60 × 10⁶ | 4.00 × 10⁵ | 2.60 × 10⁶ | 7.60 × 10⁶ |
| (inoculum) | | | | | |
| MC after | 2 | 9 | 4 | 57 | 64 |
| 1 h | | | | | |
| MC after | 0 | 0 | 1 | 3 | 0 |
| 24 h | | | | | |
| MC after | 0 | 0 | 0 | 0 | 0 |
| 72 h | | | | | |
| Log₁₀ | 6.1 | 6.2 | 5.6 | 6.4 | 6.9 |
| reduction | | | | | |

MC = microbe count

TABLE 3
Safil beige USP 2/0 coated with 5% PHMB-HCl solution

| Time | P. aeruginosa | MRSA | C. albicans | MRSE | E. coli |
|---|---|---|---|---|---|
| (days) | ATCC 9027 | ATCC 33592 | ATCC 10231 | CIP 105810 | ATCC 8739 |
| MC | 1.20 × 10⁶ | 1.60 × 10⁶ | 4.00 × 10⁵ | 2.60 × 10⁶ | 7.60 × 10⁶ |
| (inoculum) | | | | | |
| MC after | 13 | 13 | 11 | 163 | 35 |
| 1 h | | | | | |
| MC after | 0 | 0 | 0 | 2 | 0 |
| 24 h | | | | | |
| MC after | 0 | 0 | 0 | 0 | 0 |
| 72 h | | | | | |
| Log₁₀ | 6.1 | 6.2 | 5.6 | 6.4 | 6.9 |
| reduction | | | | | |

MC = microbe count

TABLE 4
Safil beige USP 2/0 coated with 1% PHMB-HCl solution

| Time | P. aeruginosa | MRSA | C. albicans | MRSE | E. coli |
|---|---|---|---|---|---|
| (days) | ATCC 9027 | ATCC 33592 | ATCC 10231 | CIP 105810 | ATCC 8739 |
| MC | 1.20 × 10⁶ | 1.60 × 10⁶ | 4.00 × 10⁵ | 2.60 × 10⁵ | 7.60 × 10⁶ |
| (inoculum) | | | | | |
| MC after | 37 | 22 | 40 | 142 | 108 |
| 1 h | | | | | |
| MC after | 0 | 0 | 0 | 5 | 0 |
| 24 h | | | | | |
| MC after | 0 | 0 | 0 | 0 | 0 |
| 72 h | | | | | |
| Log₁₀ | 6.1 | 6.2 | 5.6 | 6.4 | 6.9 |
| reduction | | | | | |

MC = microbe count

An efficient antimicrobial efficacy against Gram-positive and Gram-negative bacteria and against a blastomycete was thus detected for a coating concentration as low as 1.00 g/l of PHMB-HCl.

### 5. Cytotoxicity of PHMB-HCl

The test was carried out in accordance with the standard EN ISO/IEC 17025. The thread pieces were extracted in the absence of light with the DMEM-FBS cell culture medium at 37±2° C. for 7 days. The absorbed DMEM-FBS was made up to a volume such that the surface/volume ratio was 9 cm²/ml. As a negative control, DMEM-FBS was incubated at 37±2° C. for 7 days without test material. 7.5% v/v DMSO was used as positive control. Extracts and negative controls were diluted in five steps with DMEM-FBS solution (dilution ratio 2:3). 100 μl of the dilute solutions of the extracts and of the negative controls and also 100 μl of the positive controls were added in triplicate to wells in a porcelain plate. Thereafter, 50 μl of a freshly prepared cell suspension (7.5×10⁴ cells/ml) were added to all wells except those used for background determination. The final concentrations of the extracts were accordingly 66.7%, 44.5%, 29.6%, 19.8%, 13.2% and 8.8% v/v. The plates were then incubated at 37±2° C. in humidified air (5% CO₂/95% air) for 72±2 hours. Thereafter, the protein content of the samples was determined calorimetrically at a wavelength of 550 nm (BCA Assay method).

The test organisms used were L929 cells (DSM ACC2, mouse fibroblasts, clone of the L strand). The culture medium (Dulbecco's modified Eagle Medium, DMEM) was overlaid with 10% foetal calf serum (FBS), 100 U/ml of penicillin (P) and 100 mg/ml of streptomycin (S) DMSO was obtained from Merck (Darmstadt). FBS and P/S from Biochrom (Berlin) and BCA protein quantification kit from Interchim (France).

TABLE 5
Safil beige USP 2/0 coated with 1% PHMB solution

| Sample/dilution | Growth inhibition [%] |
|---|---|
| Positive control | 81 |
| Negative control | 0 |
| 66.7% | 10 |
| 44.5% | 5 |
| 29.6% | 3 |
| 19.8% | 3 |
| 13.2% | 2 |
| 8.8% | 0 |

Safil threads coated with solutions of higher concentration (5%, 10%, 15%) exhibit a certain cytotoxicity.

Both the antimicrobial tests and the cytotoxicity investigations show that a coating concentration of less than 5% in particular of 1% is particularly useful as coating solution.

### EXAMPLE 2

### PHMB Stearate

### 2.1 Synthesis of PHMB Stearate

In a standard stirred apparatus, 900 mL of water was stirred with 10.8 g of sodium hydroxide platelets, 88.8 g of stearic acid and with 300 mL of PHMB-HCl solution (20%). The suspensions were then heated at 80° C. for 2 h, then cooled down in an ice bath to 4° C. and filtered. Threefold resuspending in water with subsequent filtration and lyophilization overnight provided an amorphous powder which was free of impurities according to IR-spectroscopic purity determination.

2.2 Coating of Thread with PHMB Stearate

A coating range (cf. also FIG. 1) was used to continuously coat about 100 m of a Safil® beige thread of USP 2/0 gauge. The coating used was a 20% fully saturated PHMB stearate solution in toluene. The squeeze pressure of the squeeze-off rolls was 100 N, the coating speed was 5 m/min and the drying temperature was 350° C. The analytical method described in Example 1 could not be used to determine concentration.

An estimate gives a concentration of approximately 90±20 mg/m² on the thread.

The PHMB stearate is very sparingly soluble in water (<0.1 g/l) but moderately soluble in chloroform (approximately at most 30 g/l).

### 2.3 Surface Examination

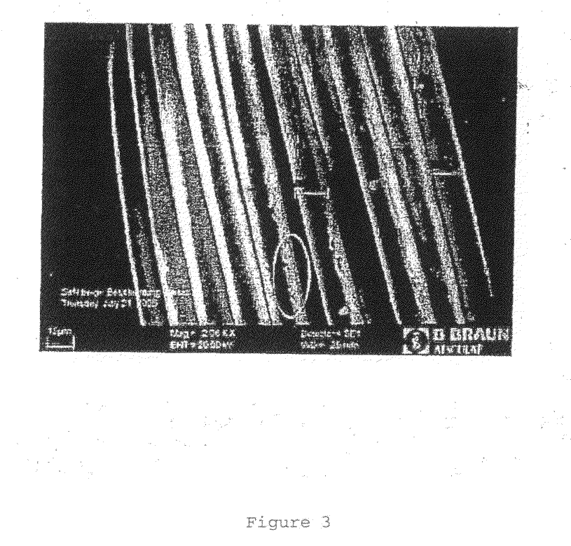

Electron micrographs were taken for surface examination. The micrographs were obtained with an acceleration voltage of 20 kV, a working distance of 25 mm and through detection of secondary electrons at 2060-fold magnification. The circles indicate coating material (cf. also FIG. 3).

The coating appears uniform. There are no grains and no deposits between the filaments (cf. also FIG. 3). Deposition of the coating is uniform.

### 2.4 Antimicrobial Efficacy

Antimicrobial efficacy was detected after 24 hours of immersion of a 10 cm long thread of the abovementioned sample in 20 mL of phosphate-buffered saline (pH=7) after inoculation with 5×10⁴ colony forming units per microbe at time t=0. The results are listed below in Table 6.

TABLE 6
Safil beige USP 2/0 coated with saturated PHMB
stearate solution

| Time | P. aeruginosa | S. aureus | C. albicans | S. epidermidis | E. coli |
|---|---|---|---|---|---|
| (days) | ATCC 9027 | ATCC 6538 | ATCC 10231 | ATCC 12228 | ATCC 8739 |
| MC | 3.50 × 10⁴ | 4.20 × 10⁴ | 3.07 × 10⁴ | 9.75 × 10⁴ | 3.47 × 10⁴ |
| (inoculum) | | | | | |
| MC after | 7.90 × 10³ | 5.00 × 10⁴ | 3.25 × 10⁴ | 8.10 × 10⁴ | 1.20 × 10⁴ |
| 0 h | | | | | |
| MC after | 1.35 × 10² | 2.65 × 10³ | 2.80 × 10³ | 5.95 × 10³ | 1.70 × 10³ |
| 24 h | | | | | |
| Log₁₀ | 2.4 | 1.2 | 1.0 | 1.2 | 1.3 |
| reduction | | | | | |

MC = microbe count

### 2.5 Cytotoxicity of PHMB Stearate

To determine the cytotoxicity, an extract of the test article was produced with the Minimum Essential Medium to which 5% foetal calf serum and 2% antibiotics (1×MEM) was added. This test extract was applied to three separate confluent monolayers of L-929 mouse fibroblasts previously propagated in humidified air (5% CO₂/95% air) at, 37±2° C. Three separate monolayers were incubated at 37° C. in the presence of 5% CO₂ for 48 hours. The monolayer in the test sample (Safil threads), in the positive control (DMSO) and in the negative control (sample without threads) were microscopically examined after 48 hours in order to ascertain changes in cell morphology. No signs of cytotoxicity were found in tests. On a scale from 0 to 4, the test material was classified 0.

0=no cytotoxicity
1=slight cytotoxicity (<20% growth inhibition)
2=medium cytotoxicity (20-50% growth inhibition)
3=moderate cytotoxicity (50-70% growth inhibition)
4=severe cytotoxicity (70-100% growth inhibition)

Table 6 reveals that the coating has a growth-inhibiting effect with regard to bacterial pathogens.

1. A biocompatible filament material having an antimicrobial finish, in particular in the form of a surficial layer, the finish comprising polyhexamethylenebiguanide (PHMB) as a nonspecifically antimicrobially active component.

2. The filament material according to claim 1, characterized in that it is in the form of a braid, a formed-loop knit or a drawn-loop knit, in particular in the form of a formed-loop knit.

3. The filament material according to claim 1, characterized in that it is in the form of suture.

4. The filament material according to claim 1, characterized in that the nonspecifically antimicrobially active component PHMB is present on the surface.

5. The filament material according to claim 1, characterized in that it is antimicrobially active for at least 7 days, preferably at least for 10 to 14 days.

6. The filament material according to claim 1, characterized in that it comprises and preferably consists of an absorbable polymer, in particular a hydrolyzable polymer.

7. The filament material according to claim 1, characterized in that the PHMB content on the surface of the coated material is in the range from 0.1 to 100 μg/cm².

8. The filament material according to claim 1, characterized in that the PHMB content in the antimicrobially finished filament material is 2-3000 ppm.

9. The filament material according to claim 1, characterized in that material has a surface coating other than the antimicrobial finish.

10. The filament material according to claim 9, characterized in that the fraction of coating material in terms of total material (filament material including coating material) is 0.1-5% by weight.

11. The filament material according to claim 9, characterized in that the coating material comprises at least one selected from the group consisting of polyethylene, polyester, silicone and polyurethane.

12. The filament material according to claim 1, characterized in that the product is free of any coating.

13. The filament material according to claim 1, characterized in that the PHMB is present in a sparingly water-soluble form.

14. The filament material according to claim 1, characterized in that the PHMB is present in the form of at least two different PHMB forms, one PHMB form preferably being sparingly water-soluble.

15. The filament material according to claim 13, characterized in that the solubility of the sparingly soluble PHMB is preferably less than 1 g/l of water, preferably less than 0.1 g/l of water.

16. A process for producing a biocompatible filament material according to claim 1, comprising the steps of:
producing an active solution from the nonspecifically antimicrobially active component PHMB and a solvent,
transferring PHMB from the active solution onto and/or into the filament material, and
drying the filament material comprising the transferred PHMB.

17. The process according to claim 16, characterized in that at least one organic solvent, especially an alcohol, ketone and/or an aromatic solvent, is used, the solvent preferably being a non-dissolver for the filament material.

18. The process according to claim 16, characterized in that water is used as solvent.

19. The process according to claim 16, characterized in that the concentration of PHMB in the active solution is in the range from 0.1% to 20% by weight and preferably in the range from 0.3% to 8% by weight.

20. The process according to claim 16, characterized in that the transferring is effected by dipping the filament material into the active solution.

21. The process according to claim 16, characterized in that the transferring is effected by spraying the active solution onto the filament material.

22. The process according to claim 16, characterized in that a coating material is dissolved together with the antimicrobially active component PHMB and subsequently applied to the filament material.

23. The process according to claim 22, characterized in that the concentration of the coating material in the active solution is in the range from 0.5% to 5% by weight and preferably in the range from 1% to 3.5% by weight.

24. The process according to claim 16, characterized in that the filament material is surface treated, in particular by sputtering or by plasma activation, in particular by plasma activation, before the active solution is applied.

25. Provision of the biocompatible filament material according to claim 1 for use in relation to the human or animal organism, especially in relation to wound closure and/or hernias.
